(19) United States
(12) Patent Application Publication
(10) Pub. No.: US 2004/0158279 A1
(43) Pub. Date: Aug. 12, 2004

(54) Loading tool

(75) Inventor: Scott R. Petersen, Brooklyn Park, MN (US)

Correspondence Address: Glenn M. Seager, CROMPTON, SEAGER & TUFTE, LLC, Suite 895, 331 Second Avenue South, Minneapolis, MN, 55401-2246, US

(73) Assignee: SCIMED LIFE SYSTEMS, INC.

(21) Appl. No.: 10/749,318

(22) Filed: Dec. 31, 2003

Related U.S. Application Data
(63) Continuation of application No. 09/976,660, filed on Oct. 10, 2001.

(51) Int. Cl. A61M 29/00

(52) U.S. Cl. 606/200

(57) ABSTRACT

The present invention relates generally to devices for filtering embolic debris from a blood vessel in conjunction with a medical procedure. More particularly, the invention includes a loading tool for loading a filter into a sheath. In addition, a method of using the loading tool to load a filter into a sheath is disclosed.

### FIELD OF THE INVENTION

The present invention relates to distal protection devices. More particularly, the present invention relates to devices for loading a distal protection filter into a delivery sheath in order to simplify delivery of the filter to an area of interest.

### BACKGROUND OF THE INVENTION

Heart disease is a major problem in the United States and throughout the world. Conditions such as atherosclerosis result in blood vessels becoming blocked or narrowed. This blockage can result in lack of oxygenation to the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices termed distal protection devices have been developed to filter out this debris.

Typical distal protection devices generally comprise a filter that is disposed on a guidewire. To facilitate delivery of the filter to an area of interest, the filter may be loaded into a delivery sheath. The sheath may then be maneuvered through the vasculature to a position downstream of a medical procedure that may generate embolic debris. The sheath may then be withdrawn from the filter and the filter may be expanded in order to capture the debris.

The filter may need to be substantially compressed within the delivery sheath in order for it to be passed through the narrow vasculature. Prepackaging of filters during manufacturing may lead to high filter deployment forces due to sheath/filter interactions and the effects of sterilization and aging of the product. A need, therefore exists, for a device that can minimize deployment forces by controlling sheath/filter interactions and the effects of sterilization and aging.

### SUMMARY OF THE INVENTION

The present invention pertains to a loading tool for loading a filter into a delivery sheath. The loading tool may help limit deployment forces by allowing a clinician to load the filter into the sheath. In addition, the loading tool may be used to load other objects including stents and balloons.

The loading tool may comprise a proximal end, a distal end, and a lumen extending therethrough. The loading tool generally tapers proximally and may include an inside diameter that is smaller near the proximal end.

The loading tool may be coupled to a delivery sheath in order to facilitate loading of the filter into the sheath. While coupled to the delivery sheath, the filter disposed proximate a distal end of an elongate shaft may be urged proximally by applying force to the elongate shaft. As the filter moves proximally, it may shift from an expanded configuration to a collapsed configuration. The filter in the collapsed configuration may be urged into a lumen of the delivery sheath. The loading tool may then be uncoupled from the delivery sheath.

### BRIEF DESCRIPTION OF THE DRAWINGS

FIG. 1 is a cross sectional view of a loading tool coupled to a delivery sheath, the loading tool having a filter disposed therein;

FIG. 2 is a cross sectional view of the loading tool coupled to the sheath, the loading tool having the filter partially collapsed and disposed therein;

FIG. 3 is a cross sectional view of the loading tool coupled to the sheath, the sheath having the filter collapsed and disposed therein;

FIG. 4 is a cross sectional view of the loading tool detached from the sheath, the sheath having the filter collapsed and disposed therein; and

FIG. 5 is a cross sectional view of an alternate loading tool coupled to the sheath, the loading tool having the filter partially collapsed and disposed therein.

### DETAILED DESCRIPTION OF THE PREFERRED EMBODIMENTS

The following description should be read with reference to the drawings wherein like reference numerals indicate like elements throughout the several views. The detailed description and drawings represent select embodiments and are not intended to be limiting.

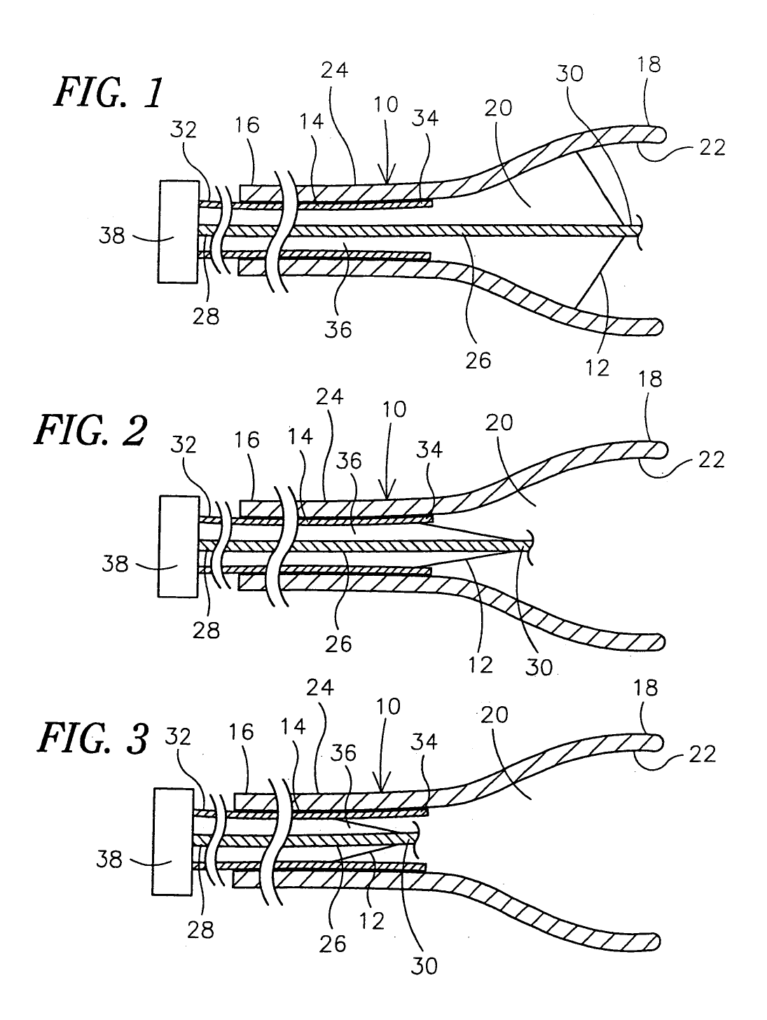

FIG. 1 is a cross sectional view of a loading tool according to a preferred embodiment of the invention. A loading tool 10 may be used to load a filter 12 into a delivery sheath 14. Loading tool 10 includes a proximal end 16, and distal end 18, and a lumen 20 extending therethrough. Loading tool 10 may be generally conical in shape and may taper at proximal end 16. Preferably, loading tool 10 has a substantially constant wall thickness and, thus, the width of lumen 20 tapers near proximal end 16. According to this embodiment, loading tool 10 has a first inside diameter region 22 near distal end 18 and a second inside diameter region 24 near proximal end 16. The inside diameter of loading tool 10 at first inside diameter region 22 is greater than the inside diameter of loading tool 10 at second inside diameter region 24. For example, the inside diameter of loading tool 10 may be about 0.08 to 0.10 inches proximate first inside diameter region 22 and may be about 0.043 to 0.08 inches proximate second inside diameter region 24.

Loading tool 10 is preferably manufactured from hypodermic tubing. Alternatively, loading tool 10 may be manufactured from materials including, but not limited to, metals, stainless steel, nickel alloys, nickel-titanium alloys, thermoplastics, high performance engineering resins, fluorinated ethylene propylene (FEP), polymer, polyethylene (PE), polypropylene (PP), polyvinylchloride (PVC), polyurethane, polytetrafluoroethylene (PTFE), polyether block amide (PEBA), polyether-ether ketone (PEEK), polyimide, polyamide, polyphenylene sulfide (PPS), polyphenylene oxide (PPO), polysufone, nylon, perfluoro(propyl vinyl ether) (PFA), and combinations thereof.

Manufacturing of loading tool 10 may include flaring one end of hypodermic or another type of tubing. For example, extruded tubing with an inside diameter of about 0.043 inches may be flared at one end. Alternatively, a portion of a tube may be necked down over a mandrel. For example, an extruded tube may be necked down over a mandrel having an outside diameter of about 0.043 inches. In an second alternative, loading tool 10 may be manufactured by molding material to the desired shape.

Filter 12 is coupled to a shaft 26 having a proximal end 28 and a distal end 30. Filter 12 is coupled to shaft 26 proximate distal end 30. In addition, filter 12 may include a plurality of ribs or struts 25 that bridge filter 12 and shaft 26 and may help to support and/or collapse filter 12. Shaft 26 may be a guidewire and is preferably comprised of for example, metals including stainless steel, nickel alloys, and nickel-titanium alloys.

Filter 12 operates between a closed collapsed profile, adapted for insertion into delivery sheath 14, and an open radially-expanded deployed profile for collecting debris in a body lumen. Filter 12 may include a collapsible proximally-tapered frame having a mouth and a plurality of longitudinally-extending ribs. In an expanded profile, the mouth is opened and the ribs extend radially outwardly to support the mouth.

Filter 12 may be generally cone-shaped, and have a proximal and a distal end. The distal end is a narrow, “V”-shaped end and is preferably fixedly secured or formed to shaft 26. The proximal end has a relatively wide opening. Alternatively, filter 12 may be cylindrical with a relatively rounded distal end.

Filter 12 may include a filtering mesh formed of a polymer membrane and including a plurality of small openings. For example, filter 12 may be constructed of a polyurethane sheet, and the openings may be formed in the polyurethane sheet by known laser techniques. Holes or openings are sized to allow blood flow therethrough but restrict flow of debris or emboli floating in the body lumen or cavity.

Delivery sheath 14 has a proximal end 32, a distal end 34, and a lumen 36 extending therethrough. Shaft 26 can be disposed within lumen 36. Delivery sheath 14 may be comprised of metals similar to those listed above for loading tool 10.

Proximal end 28 of shaft 26 and proximal end 32 of delivery sheath 14 may be coupled to a manifold 38. Manifold 38 may include means for securing shaft 26 relative to delivery sheath 14. Securing shaft 26 relatively to delivery sheath 14 may allow delivery sheath 14 to be urged proximally in order to deploy filter 12.

Shaft 26, delivery sheath 14, and filter 12 may be disposed within a package 39. Package 39 may be sterile and may be an appropriate configuration for delivery of the product to a clinician. Additionally, loading tool 10 may be disposed within package 39. According to this embodiment, use of loading tool 10 may include the steps of removing shaft 26, delivery sheath 14, filter 12, and loading tool 10 from package 39. Then filter 12 would be placed in sheath 14.

FIG. 2 is a cross sectional view of loading tool 10 coupled to delivery sheath 20, wherein filter 12 is partially collapsed within loading tool 10. Filter 12 may be urged proximally by applying force to shaft 26 or other suitable means. When filter 12 moves proximally, due to this force, the position of filter 12 relative to loading tool 10 shifts to a region (e.g., second inside diameter region 24) where the inside diameter within loading tool 10 becomes smaller. This results in a partial collapse of filter 12. According to this embodiment, as filter 12 moves proximally, it may shift from an expanded configuration to a collapsed configuration.

Loading tool 10 is adapted to be coupled to delivery sheath 14. For example, loading tool 10 may be coupled to delivery sheath 14 by a friction fit. According to this embodiment, at least a portion of loading tool 10 is disposed over delivery sheath 14. Loading tool 10 may be uncoupled (i.e., separated) from delivery sheath 14 by applying force in opposing directions to each element. Alternative ways of coupling loading tool 10 to sheath 14 may be used without departing from the spirit of the invention. For example, adhesives, heat bonds, mechanical fittings, luer fitting, and alternative means may be used.

FIG. 3 depicts filter 12 collapsed and disposed within delivery sheath 14. When filter 12 reaches a position proximate second inside diameter region 24 of loading tool 10, filter 12 may be suitably collapsed for entry into lumen 36 of delivery sheath 14. Filter 12 may be urged into lumen 36 by applying force to shaft 26 or other suitable means.

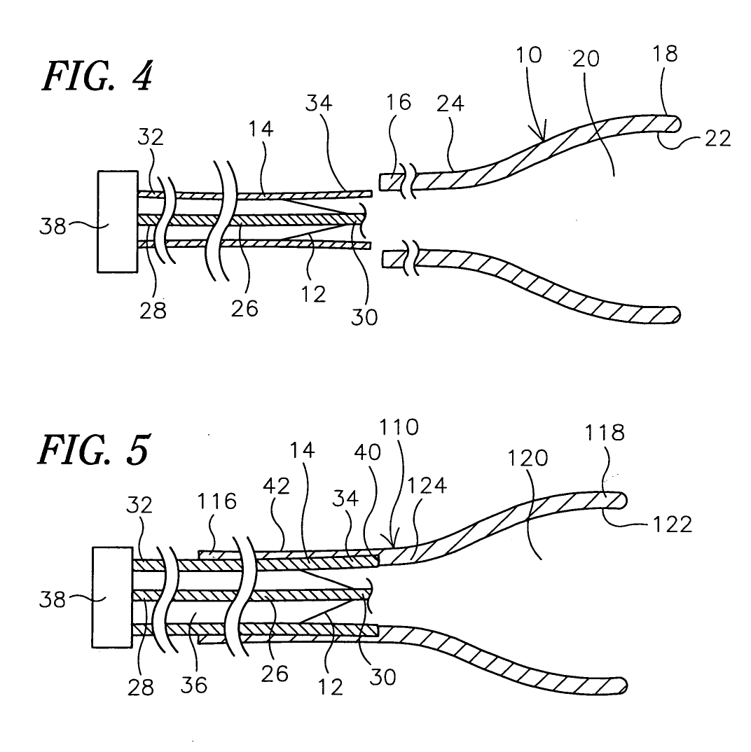

FIG. 4 is a plan overview of loading tool 10 detached from delivery sheath 14. When filter 12 is collapsed and disposed within lumen 36 of delivery sheath 14, loading tool 10 may be uncoupled from delivery sheath 14. Uncoupling of loading tool 10 from delivery sheath results in filter 12 being appropriately prepared for entry into a blood vessel (e.g., the vasculature of a patient).

FIG. 5 is plan overview of an alternate loading tool. Loading tool 110 is substantially similar to loading tool 10 except that it further comprises a notched region 40 defining a third inside diameter region 42. Loading tool 110 includes proximal end 116, distal end 118, lumen 120 extending therethrough, first inside diameter region 122, and second inside diameter region 124. Preferably, the inside diameter of loading tool 110 at third inside diameter region 42 is greater than the inside diameter of loading tool 110 at second inside diameter region 124. For example the third inside diameter may be substantially equal to the outside diameter of delivery sheath 14.

Notched region 40 may provide a smooth transition between inside diameters of loading tool 110 and delivery sheath 14. According to this embodiment, the inside diameter of loading tool 110 at third inside diameter region 42 may be substantially equal to the outside diameter of delivery sheath 14. Therefore, loading tool 110 may be coupled to delivery sheath 14 by disposing third inside diameter region 42 over delivery sheath 14. In addition, the inside diameter of loading tool 110 at second inside diameter region 124 may be substantially equal to the inside diameter of delivery sheath 14. Therefore, filter 12 may easily move from lumen 120 of loading tool 110 into lumen 36 of sheath 14.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A system for loading a distal protection device, comprising:
a delivery sheath having a proximal end, a distal end, and a lumen extending though at least a portion of the distal end;
an elongate shaft having a proximal end and a distal end, wherein at least a portion of the shaft is adapted to be disposed within the lumen of the delivery sheath;
a filter disposed proximate the distal end of the shaft; and
a loading tool for loading the filter into the delivery sheath, the loading tool having a proximal end, a distal end, and a lumen extending therethrough adapted to receive at least a portion of the filter, wherein at least a portion of the loading tool is configured to be coupled to the delivery sheath.

2. The system in accordance with claim 1, wherein the sheath, the shaft, the filter, and the loading tool are disposed within a one or more sterile packages.

3. A kit for loading a distal protection device, comprising:
a delivery sheath having a proximal end, a distal end, and a lumen extending though at least a portion of the distal end;
an elongate shaft having a proximal end and a distal end, wherein at least a portion of the shaft is adapted to be disposed within the lumen of the delivery sheath;
a filter disposed proximate the distal end of the shaft;
wherein the sheath, the shaft, and the filter are disposed within one or more packages; and
a loading tool for loading the filter into the delivery sheath, the loading tool having a proximal end, a distal end, and a lumen extending therethrough adapted to receive at least a portion of the filter, wherein at least a portion of the loading tool is configured to be coupled to the delivery sheath.

4. The kit in accordance with claim 3, wherein the loading tool is disposed within the package.

5. The kit in accordance with claim 3, wherein the package is sterile.

6. A method of loading a filter, comprising the steps of:
providing a sterile package including a delivery sheath, an elongate shaft having a filter disposed proximate a distal end thereof, and a loading tool;
removing the delivery sheath from the sterile package;
removing the loading tool from the sterile package;
coupling the loading tool to a delivery sheath;
removing the shaft from the sterile package;
loading the filter within the delivery sheath by applying force to the elongate shaft, wherein the filter passes through a lumen within the loading tool and into a lumen within the delivery sheath; and
separating the loading tool from the delivery sheath.

7. A system for loading a distal protection device, comprising:
a delivery sheath having a proximal end, a distal end, and a lumen extending though at least a portion of the distal end;
an elongate shaft having a proximal end and a distal end, wherein at least a portion of the shaft is adapted to be disposed within the lumen of the delivery sheath;
a filter disposed proximate the distal end of the shaft; and
a loading tool for loading the filter into the delivery sheath, the loading tool having a proximal end, a distal end, and a lumen extending therethrough adapted to receive at least a portion of the filter, wherein at least a portion of the loading tool is adapted to be coupled to the delivery sheath.

8. The system for loading a filter in accordance with claim 7, wherein the loading tool further comprises a first inside diameter region proximate the distal end thereof.

9. The system for loading a filter in accordance with claim 8, wherein the loading tool further comprises a second inside diameter region proximate the proximal end thereof.

10. The system for loading a filter in accordance with claim 9, wherein the inside diameter of the loading tool at the first outside diameter region is greater than the inside diameter of the loading tool at the second outside diameter region.

11. The system for loading a filter in accordance with claim 10, wherein the inside diameter of the loading tool at the first outside diameter region is about 0.080 to 0.100 inches.

12. The system for loading a filter in accordance with claim 10, wherein the inside diameter of the loading tool at the second outside diameter region is about 0.043 to 0.080 inches.

13. The system for loading a filter in accordance with claim 9, wherein the loading tool further comprises a notched region and a third inside diameter region.

14. The system for loading a filter in accordance with claim 13, wherein the delivery sheath further comprises an outside diameter and an inside diameter.

15. The system for loading a filter in accordance with claim 14, wherein the inside diameter of the loading tool at the third inside diameter region and the outside diameter of the delivery sheath are substantially equal.

16. The system for loading a filter in accordance with claim 15, wherein the inside diameter of the loading tool at the second inside diameter region and the inside diameter of the delivery sheath are substantially equal.

17. The system for loading a filter in accordance with claim 7, wherein the filter may be in an expanded configuration.

18. The system for loading a filter in accordance with claim 17, wherein the filter is in the expanded condition when it is disposed within the lumen of the loading tool near the distal end thereof.

19. The system for loading a filter in accordance with claim 7, wherein the filter may be in a collapsed configuration.

20. The system for loading a filter in accordance with claim 19, wherein the filter is in the expanded condition when it is disposed within the lumen of the loading tool near the proximal end thereof.

21. A method of loading a filter, comprising the steps of:
providing a loading tool having a proximal end, a distal end, and a lumen extending therethrough;
providing an elongate shaft having a filter disposed proximate a distal end thereof, wherein the filter is configured to be disposed with the lumen of the loading tool and in an expanded configuration;
coupling the loading tool to a delivery sheath having a proximal end, a distal end, and a lumen extending through at least a portion of the distal end;
urging the filter toward the proximal end of the loading tool by applying force to the elongate shaft, wherein the filter shifts from the expanded configuration to a collapsed configuration; and
urging the filter to within the lumen of the delivery sheath.

22. The method in accordance with claim 21, wherein the loading tool further comprises a first inside diameter region proximate the distal end thereof.

23. The method in accordance with claim 22, wherein the loading tool further comprises a second inside diameter region proximate the proximal end thereof.

24. The method in accordance with claim 23, wherein the inside diameter of the loading tool at the first outside diameter region is greater than the inside diameter of the loading tool at the second outside diameter region.

25. The method in accordance with claim 24, wherein the inside diameter of the loading tool at the first outside diameter region is about 0.080 to 0.100 inches.

26. The method in accordance with claim 24, wherein the inside diameter of the loading tool at the second outside diameter region is about 0.043 to 0.080 inches.

27. The method in accordance with claim 23, wherein the loading tool further comprises a notched region and a third inside diameter region.

28. The method in accordance with claim 27, wherein the delivery sheath further comprises an outside diameter and an inside diameter.

29. The method in accordance with claim 28, wherein the inside diameter of the loading tool at the third inside diameter region and the outside diameter of the delivery sheath are substantially equal.

30. The method in accordance with claim 28, wherein the inside diameter of the loading tool at the second inside diameter region and the inside diameter of the delivery sheath are substantially equal.

31. The method in accordance with claim 21, further comprising the step of uncoupling the loading tool from the delivery sheath.

32. The method in accordance with claim 31, wherein the step of uncoupling the loading tool from the delivery sheath results in the filter being appropriately prepared for entry into a blood vessel.
